(19) United States
(12) Patent Application Publication
(10) Pub. No.: US 2022/0096019 A1
(43) Pub. Date: Mar. 31, 2022

(54) ELECTRONIC DEVICE FOR SIGNAL INTERFERENCE COMPENSATION

(71) Applicant: Industrial Technology Research Institute, Hsinchu (TW)

(72) Inventors: Cheng-Hung Yu, Taoyuan City (TW); Shuen-Yu Yu, New Taipei City (TW); Heng-Yin Chen, Hsinchu County (TW)

(73) Assignee: Industrial Technology Research Institute, Hsinchu (TW)

(21) Appl. No.: 17/144,151

(22) Filed: Jan. 8, 2021

(30) Foreign Application Priority Data
Sep. 25, 2020 (TW) 109133221

(51) Int. Cl. A61B 5/00; A61B 5/389; A61B 5/304; A61B 5/313

(52) U.S. Cl. CPC A61B 5/7225; A61B 5/313; A61B 5/304; A61B 5/389

(57) ABSTRACT

An electronic device for signal interference compensation is provided. The first signal line is electrically connected to the transmitter. The second signal line is electrically connected to the receiver and coupled with the first signal line. The electrode is electrically connected to the second signal line and measures a physiological signal. The processor is electrically connected to the transmitter and the receiver, and configured to: transmit, via the transmitter, an active signal to the first signal line; receive, via the receiver, a coupling signal corresponding to the active signal from the second signal line, and calculate a compensation value according to the coupling signal; and receive, via the receiver, an interfered signal corresponding to the physiological signal, and restore the physiological signal according to the compensation value and the interfered signal in response to the compensation value matching the interfered signal.

### CROSS-REFERENCE TO RELATED APPLICATION

This application claims the priority benefit of Taiwan application serial no. 109133221, filed on Sep. 25, 2020. The entirety of the above-mentioned patent application is hereby incorporated by reference herein.

### TECHNICAL FIELD

The disclosure relates to an electronic device for signal interference compensation.

### BACKGROUND

Smart fabrics may be used in clothes. When a person wears clothes including smart fabrics, the smart fabrics are able to measure an electromyogram (EMG) signal from the skin of this person, and make use of the EMG signal, such as using the EMG signal to determine a muscle tiredness level of the human body. Under various considerations such as aesthetics, comfort, etc., a plurality of smart fabrics often need to be wound together in the applications of smart fabrics. In such case, however, the EMG signals measured by the smart fabrics may be distorted under the influence of crosstalk. The power of EMG signals is usually small. Therefore, the crosstalk may result in an excessive error in the measurement of EMG signals, making it difficult to accurately determine the muscle tiredness level according to the EMG signals that are measured.

### SUMMARY

An electronic device for signal interference compensation according to an embodiment of the disclosure includes a transmitter, a first signal line, a receiver, a second signal line, an electrode, and a processor. The first signal line is electrically connected to the transmitter. The second signal line is electrically connected to the receiver and coupled with the first signal line. The electrode is electrically connected to the second signal line and measures a physiological signal. The processor is electrically connected to the transmitter and the receiver, and configured to: transmit, via the transmitter, an active signal to the first signal line; receive, via the receiver, a coupling signal corresponding to the active signal from the second signal line, and calculate a compensation value according to the coupling signal; and receive, via the receiver, an interfered signal corresponding to the physiological signal, and restore the physiological signal according to the compensation value and the interfered signal in response to the compensation value matching the interfered signal.

An electronic device for signal interference compensation according to an embodiment of the disclosure includes a transmitter, a first signal line, a first receiver, a second signal line, a second receiver, a third signal line, an electrode, and a processor. The first signal line is electrically connected to the transmitter. The second signal line is electrically connected to the first receiver and coupled with the first signal line. The third signal line is electrically connected to the second receiver and coupled with the first signal line. The electrode is electrically connected to the third signal line and measures a physiological signal. The processor is electrically connected to the transmitter, the first receiver, and the second receiver, and is configured to: transmit, via the transmitter, an active signal to the first signal line; receive, via the first receiver, a coupling signal corresponding to the active signal from the second signal line, and calculate a compensation value according to the coupling signal; and receive, via the second receiver, an interfered signal corresponding to the physiological signal, and restore the physiological signal according to the compensation value and the interfered signal in response to the compensation value matching the interfered signal.

An electronic device for signal interference compensation according to an embodiment of the disclosure includes a first transceiver, a first signal line, a second transceiver, a second signal line, a first electrode, and a processor. The first signal line is electrically connected to the first transceiver. The second signal line is electrically connected to the second transceiver and coupled to the first signal line. The first electrode is electrically connected to the second signal line and measures a physiological signal. The processor is electrically connected to the first transceiver and the second transceiver, and is configured to: transmit, via the first transceiver, an active signal to the first signal line; receive, via the second transceiver, a coupling signal corresponding to the active signal from the second signal line, and calculate a compensation value according to the coupling signal; and receive, via the second transceiver, an interfered signal corresponding to the physiological signal, and restore the physiological signal according to the compensation value and the interfered signal in response to the compensation value matching the interfered signal.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a schematic diagram illustrating an electronic device for signal interference compensation according to an embodiment of the disclosure.

FIG. 2 is a schematic diagram illustrating obtaining a compensation value and restoring a physiological signal by an electronic device according to an embodiment of the disclosure.

FIGS. 3A and 3B are schematic diagrams illustrating measuring a physiological signal after obtaining all compensation values according to an embodiment of the disclosure.

FIGS. 4A and 4B are schematic diagrams illustrating measuring a physiological signal while obtaining a plurality of compensation values according to an embodiment of the disclosure.

FIG. 5 is a schematic diagram illustrating an electronic device for signal interference compensation according to an embodiment of the disclosure.

FIG. 6 is a schematic diagram illustrating obtaining a compensation value and restoring a physiological signal by an electronic device according to an embodiment of the disclosure.

FIGS. 7A and 7B are schematic diagrams illustrating obtaining a compensation value while simultaneously measuring a physiological signal according to an embodiment of the disclosure.

FIG. 8 is a schematic diagram illustrating an electronic device for signal interference compensation according to an embodiment of the disclosure.

FIG. 9 is a schematic diagram illustrating obtaining a compensation value and restoring a physiological signal by an electronic device according to an embodiment of the disclosure.

FIG. 10 is a flowchart illustrating the first method for signal interference compensation according to an embodiment of the disclosure.

FIG. 11 is a flowchart illustrating the second method for signal interference compensation according to an embodiment of the disclosure.

FIG. 12 is a flowchart illustrating the third method for signal interference compensation according to an embodiment of the disclosure.

### DETAILED DESCRIPTION OF DISCLOSED EMBODIMENTS

In order to measure a physiological signal of a user in a real-time manner, such as measuring an EMG signal, an electrocardiography (ECG) signal, an electroencephalography (EEG) signal, signal lines of smart fabrics may be sewed into the clothes which the user wears. The signal line may measure the physiological signal of the user by contacting the user's skin. However, when multiple signal lines are wound together, a crosstalk may occur between signal lines, and the signal lines may affect one another. As a result, the physiological signals transmitted by the signal lines may be distorted. In order to reduce the influence of crosstalk on the physiological signal, an embodiment of the disclosure provides a method for restoring the physiological signal through signal interference compensation.

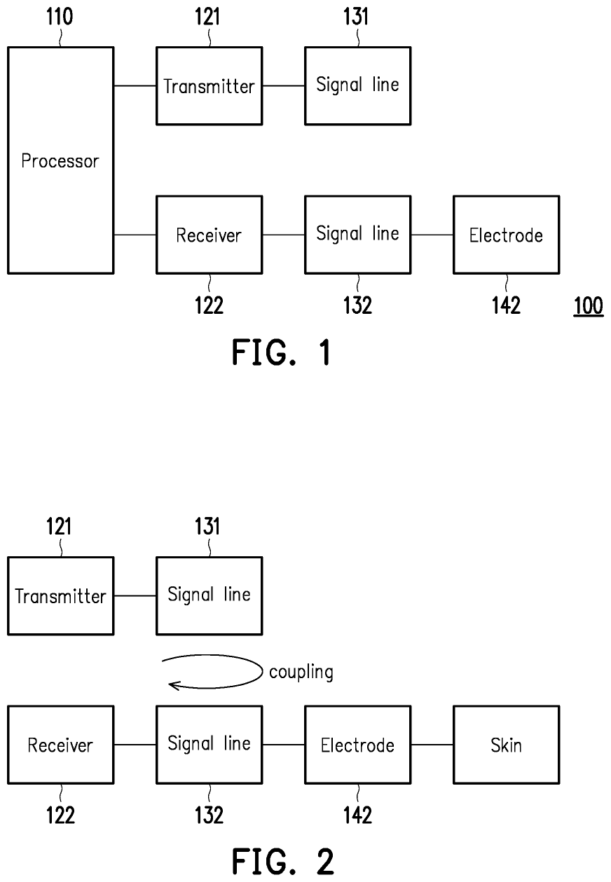

FIG. 1 is a schematic diagram illustrating an electronic device 100 for signal interference compensation according to an embodiment of the disclosure. The electronic device 100 may include a processor 110, a transmitter 121, a receiver 122, a signal line 131, a signal line 132, and an electrode 142.

The processor 110 is, for example, a central processing unit (CPU), or other programmable general-purpose/specific purpose micro control units (MCUs), microprocessors, digital signal processors (DSPs), programmable controllers, application specific integrated circuits (ASICs), graphics processing units (GPUs), image signal processors (ISPs), image processing units (IPUs), arithmetic logic units (ALUs), complex programmable logic devices (CPLDs), field programmable gate arrays (FPGAs), other similar components, or a combination thereof. The processor 110 may be electrically connected to the transmitter 121 and the receiver 122.

The processor 110 includes a storage medium. The storage medium may be, for example, any type of fixed or removable random access memories (RAMs), read-only memories (ROMs), flash memories, hard disk drives (HDDs), solid state drives (SSDs), other similar components, or a combination thereof.

The transmitter 121 may be electrically connected to the signal line 131, and may be configured to transmit a signal to the signal line 131. The transmitter 121 may be further capable of, for example, low noise amplification, impedance matching, frequency mixing, up or down frequency conversion, filtering, amplification, and similar operations.

The receiver 122 may be electrically connected to the signal line 132, and may be configured to receive a signal from the signal line 132. The receiver 122 may be further capable of, for example, low noise amplification, impedance matching, frequency mixing, up or down frequency conversion, filtering, amplification, and similar operations.

The electrode 142 may be electrically connected with the signal line 132. When contacting the user's skin, the electrode 142 may measure a physiological signal from the skin. The physiological signal is, for example, an EMG signal.

FIG. 2 is a schematic diagram illustrating obtaining a compensation value and restoring the physiological signal by the electronic device 100 according to an embodiment of the disclosure. The signal line 132 may be wound together with the signal line 131, and may be coupled with the signal line 131. The processor 110 may transmit an active signal at a specific frequency to the signal line 131 via the transmitter 121. The signal line 132 wound with the signal line 131 may generate a coupling signal corresponding to the active signal. The processor 110 may receive the coupling signal via the receiver 122, and calculate a compensation value according to the coupling signal.

The processor 110 may obtain a plurality of compensation values in correspondence with different frequencies or different frequency ranges, so as to generate a compensation table according to the compensation values. Specifically, the active signal may include a first signal corresponding to a first frequency (or a first frequency range) and a second signal corresponding to a second frequency (or a second frequency range). The processor 110 may transmit the first signal to the signal line 131 via the transmitter 121 in a first time period. The signal line 132 may generate a first coupling signal in correspondence with the first signal. The processor 110 may receive the first coupling signal via the receiver 122, and calculate a first compensation value according to the first signal and the first coupling signal. In addition, the first compensation value corresponds to the first frequency. Besides, the processor 110 may transmit the second signal to the signal line 131 via the transmitter 121 in a second time period different from the first time period. The signal line 132 may generate a second coupling signal in correspondence with the second signal. The processor 110 may receive the second coupling signal via the receiver 122, and calculate a second compensation value according to the second signal and the second coupling signal. The second compensation value corresponds to the second frequency different from the first frequency. The compensation value may include a ratio between the coupling signal and the active signal. For example, as shown in Formula (1), A(f) corresponds to an active signal at a frequency f (or a frequency range f), B(f) corresponds to a coupling signal corresponding to the frequency f, and W(f) is a compensation value corresponding to the frequency f. In an embodiment, the frequency f (or the frequency range f) may range between a frequency of 5 Hz to a frequency of 1500 Hz.

$$W ( f ) = \frac{B ( f )}{A ( f )} \qquad ( 1 )$$

The processor 110 may obtain N compensation values respectively corresponding to different frequencies (or frequency ranges), and generate a compensation table as shown in Table 1 according to the N compensation values, N being a positive integer. If the storage medium of the processor 110 already stores an existing compensation table, the processor 110 may update the existing compensation table according to the N compensation values.

TABLE 1

| Frequency (or | | | | | |
|---|---|---|---|---|---|
| frequency range) | f1 | f2 | f3 | . . . | fN |
| Active signal | A(f1) | A(f2) | A(f3) | . . . | A(fN) |
| Coupling signal | B(f1) | B(f2) | B(f3) | . . . | B(fN) |
| Compensation value | W(f1) | W(f2) | W(f3) | . . . | W(fN) |

After obtaining the compensation table, the processor 110 may use the compensation table to restore the measured physiological signal. Specifically, the electrode 142 may contact the user's skin to measure the physiological signal. The physiological signal may be transmitted to the receiver 122 via the signal line 132. However, under the influence of crosstalk, the physiological signal may be turned into an interfered signal when passing through the signal line 132. After the processor 110 receives the interfered signal via the receiver 122, the processor 110 may restore the physiological signal measured by the electrode 142 according to the compensation value and the interfered signal in response to the compensation value matching the interfered signal in the compensation table.

Specifically, the processor 110 may detect the frequency of the interfered signal, and choose a compensation value matching the frequency of the interfered signal from the compensation table to compensate the interfered signal and thereby generate the physiological signal. For example, if the interfered signal includes a signal corresponding to the frequency f1 and a signal corresponding to the frequency f2, the processor 110 may compensate the signal corresponding to the frequency f1 in the interfered signal according to the compensation value W(f1) corresponding to the frequency f1, thereby restoring the signal corresponding to the frequency f1 in the physiological signal. The processor 110 may also compensate the signal corresponding to the frequency f2 in the interfered signal according to the compensation value W(f2) corresponding to the frequency f2, thereby restoring the signal corresponding to the frequency f2 in the physiological signal. The processor 110 may generate a restored physiological signal according to Formula (2) in the following, where W(f) is a compensation value corresponding to the frequency f, X(f) is a signal corresponding to the frequency f in the physiological signal, and Y(f) is a signal corresponding to the frequency f in the interfered signal.

$$X ( f ) = \frac{Y ( f )}{1 - W ( f )} \qquad ( 2 )$$

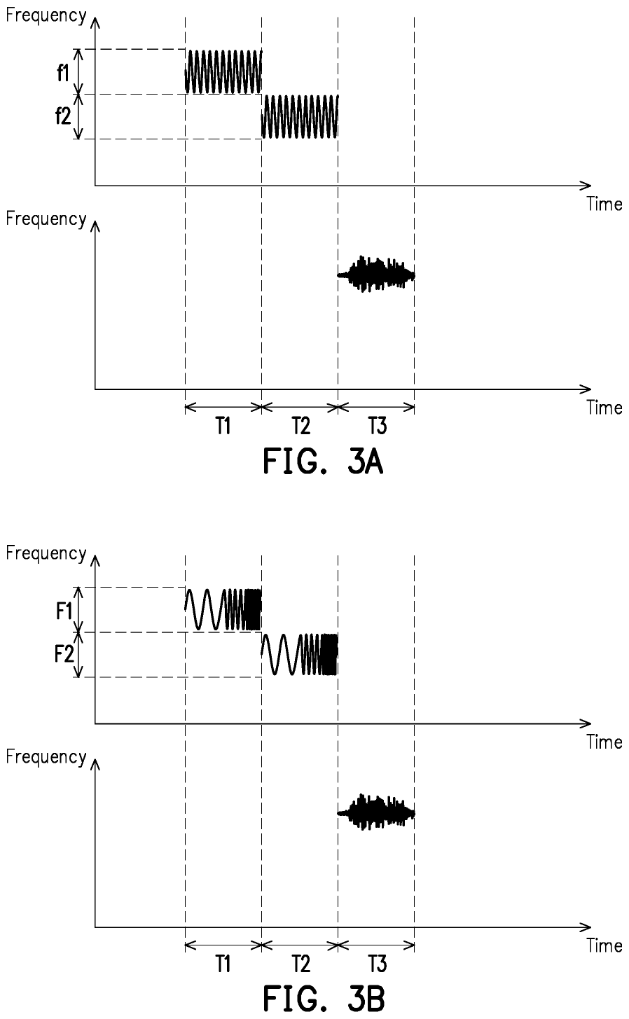

In an embodiment, the processor 110 may start receiving the interfered signal to measure the physiological signal after receiving a complete compensation table (or finishing updating the compensation table). FIGS. 3A and 3B are schematic diagrams illustrating measuring a physiological signal after obtaining all compensation values according to an embodiment of the disclosure. Referring to FIG. 3A, the processor 110 may transmit an active signal corresponding to the frequency f1 to the signal line 131 via the transmitter 121 in a time period T1, so as to obtain the compensation value corresponding to the frequency f1. Similarly, the processor 110 may transmit an active signal corresponding to the frequency f2 to the signal line 131 via the transmitter 121 in a time period T2, so as to obtain the compensation value corresponding to the frequency C. After obtaining the compensation value corresponding to the frequency f1 and the compensation value corresponding to the frequency f2, the processor 110 may receive the interfered signal via the receiver 122 in a time period T3. The interfered signal corresponds to the physiological signal measured by the electrode 142, and the time period T3 may be after the time period T1 and the time period T2. In an embodiment, the frequency f1 or the frequency f2 may be 5 Hz, 6 Hz, 7 Hz, . . . , or 1500 kHz, etc., and the frequency f1 and the frequency C may be different from each other.

Referring to FIG. 3B, the processor 110 may transmit an active signal corresponding to a frequency range F1 to the signal line 131 via the transmitter 121 in the time period T1, so as to obtain the compensation value corresponding to the frequency range F1. Similarly, the processor 110 may transmit an active signal corresponding to a frequency range F2 to the signal line 131 via the transmitter 121 in the time period T2, so as to obtain the compensation value corresponding to the frequency range F2. After obtaining the compensation value corresponding to the frequency range F1 and the compensation value corresponding to the frequency range F2, the processor 110 may receive the interfered signal via the receiver 122 in the time period T3. The interfered signal corresponds to the physiological signal measured by the electrode 142, and the time period T3 may be after the time period T1 and the time period T2. In an embodiment, the frequency range F1 or the frequency range F2 may be a frequency range between 5 Hz to 1500 kHz, and the frequency range F1 and the frequency range F2 may be different from each other.

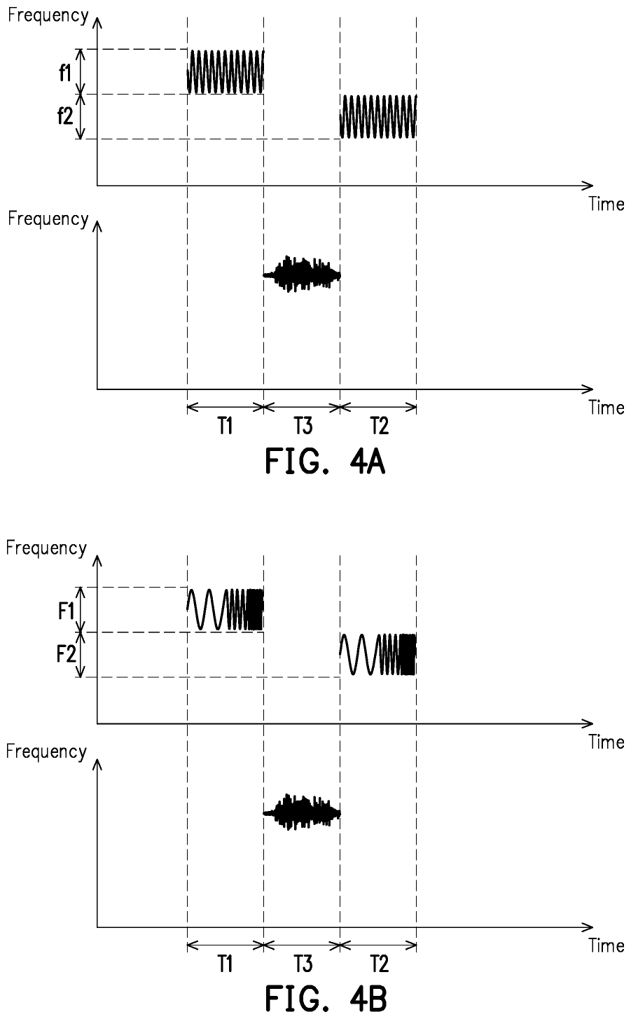

In an embodiment, the processor 110 may receive the interfered signal to measure the physiological signal while receiving (or updating) the compensation values of the compensation table. Therefore, the processor 110 may measure the physiological signal when the generation or updating of the compensation table has not been finished, thereby reducing the waiting time required in measuring the physiological signal. If a compensation value corresponding to a specific frequency has not been updated, the processor 110 may restore the physiological signal according to an existing compensation value corresponding to the specific frequency. FIGS. 4A and 4B are schematic diagrams illustrating measuring a physiological signal while obtaining a plurality of compensation values according to an embodiment of the disclosure. Referring to FIG. 4A, the processor 110 may transmit an active signal corresponding to the frequency f1 to the signal line 131 via the transmitter 121 in the time period T1, so as to obtain the compensation value corresponding to the frequency f1. Similarly, the processor 110 may transmit an active signal corresponding to the frequency f2 to the signal line 131 via the transmitter 121 in the time period T2, so as to obtain the compensation value corresponding to the frequency f2. The processor 110 may receive the interfered signal via the receiver 122 in the time period T3. The interfered signal corresponds to the physiological signal measured by the electrode 142, and the time period T3 may be between the time period T1 and the time period T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 4B, the processor 110 may transmit an active signal corresponding to the frequency range F1 to the signal line 131 via the transmitter 121 in the time period T1, so as to obtain the compensation value corresponding to the frequency range F1. Similarly, the processor 110 may transmit an active signal corresponding to the frequency range F2 to the signal line 131 via the transmitter 121 in the time period T2, so as to obtain the compensation value corresponding to the frequency range F2. The processor 110 may receive the interfered signal via the receiver 122 in the time period T3. The interfered signal corresponds to the physiological signal measured by the electrode 142, and the time period T3 may be between the time period T1 and the time period T2. In an embodiment, the frequency range F1 or the frequency range F2 may be a frequency range between 5 Hz to 1500 kHz, and the frequency range F1 and the frequency range F2 may be different from each other.

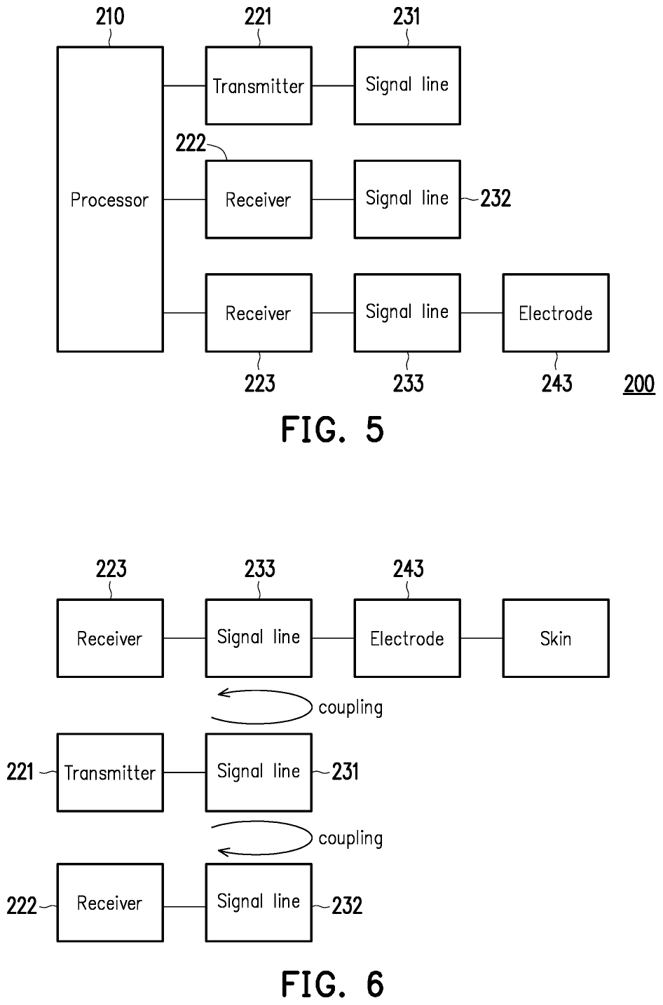

FIG. 5 is a schematic diagram illustrating an electronic device 200 for signal interference compensation according to an embodiment of the disclosure. The electronic device 200 may include a processor 210, a transmitter 221, a receiver 222, a receiver 223, a signal line 231, a signal line 232, a signal line 233, and an electrode 243.

The processor 210 is, for example, a central processing unit (CPU), or other programmable general-purpose/specific purpose micro control units (MCUs), microprocessors, digital signal processors (DSPs), programmable controllers, application specific integrated circuits (ASICs), graphics processing units (GPUs), image signal processors (ISPs), image processing units (IPUs), arithmetic logic units (ALUs), complex programmable logic devices (CPLDs), field programmable gate arrays (FPGAs), other similar components, or a combination thereof. The processor 210 may be electrically connected to the transmitter 221, the receiver 222, and the receiver 223. The processor 210 may include a storage medium. The storage medium may be, for example, any type of fixed or removable random access memories (RAMs), read-only memories (ROMs), flash memories, hard disk drives (HDDs), solid state drives (SSDs), other similar components, or a combination thereof.

The transmitter 221 may be electrically connected to the signal line 231, and may be configured to transmit a signal to the signal line 231. The transmitter 221 may be further capable of, for example, low noise amplification, impedance matching, frequency mixing, up or down frequency conversion, filtering, amplification, and similar operations.

The receiver 222 may be electrically connected to the signal line 232, and may be configured to receive a signal from the signal line 232. The receiver 222 may be further capable of, for example, low noise amplification, impedance matching, frequency mixing, up or down frequency conversion, filtering, amplification, and similar operations.

The receiver 223 may be electrically connected to the signal line 233, and may be configured to receive a signal from the signal line 233. The receiver 223 may be further capable of, for example, low noise amplification, impedance matching, frequency mixing, up or down frequency conversion, filtering, amplification, and similar operations.

The electrode 243 may be electrically connected with the signal line 233. When contacting the user's skin, the electrode 243 may measure a physiological signal from the skin.

FIG. 6 is a schematic diagram illustrating obtaining a compensation value and restoring the physiological signal by the electronic device 200 according to an embodiment of the disclosure. The signal line 232 may be wound together with the signal line 231, and may be coupled with the signal line 231. The signal line 233 may be wound together with the signal line 231, and may be coupled with the signal line 231. The processor 210 may transmit an active signal at a specific frequency to the signal line 231 via the transmitter 221. The signal line 232 wound with the signal line 231 may generate a coupling signal corresponding to the active signal. The processor 210 may receive the coupling signal via the receiver 222, and calculate a compensation value according to the coupling signal.

The processor 210 may obtain a plurality of compensation values in correspondence with different frequencies or different frequency ranges, so as to generate a compensation table according to the compensation values. Specifically, the active signal may include the first signal corresponding to the first frequency (or the first frequency range) and the second signal corresponding to the second frequency (or the second frequency range). The processor 210 may transmit the first signal to the signal line 231 via the transmitter 221 in the first time period. The signal line 232 may generate the first coupling signal in correspondence with the first signal. The processor 210 may receive the first coupling signal via the receiver 222, and calculate the first compensation value according to the first signal and the first coupling signal. In addition, the first compensation value corresponds to the first frequency. Besides, the processor 210 may transmit the second signal to the signal line 231 via the transmitter 221 in the second time period different from the first time period. The signal line 232 may generate the second coupling signal in correspondence with the second signal. The processor 210 may receive the second coupling signal via the receiver 222, and calculate the second compensation value according to the second signal and the second coupling signal. The second compensation value corresponds to the second frequency different from the first frequency. The processor 210 may obtain N compensation values respectively corresponding to different frequencies (or frequency ranges), and generate the compensation table as shown in Table 1 according to the N compensation values. If the storage medium of the processor 210 already stores an existing compensation table, the processor 210 may update the existing compensation table according to the N compensation values.

After obtaining the compensation table, the processor 210 may use the compensation table to restore the measured physiological signal. Specifically, the electrode 243 may contact the user's skin to measure the physiological signal. The physiological signal may be transmitted to the receiver 223 via the signal line 233. However, under the influence of crosstalk, the physiological signal may be turned into an interfered signal when passing through the signal line 233.

After the processor 210 receives the interfered signal via the receiver 223, the processor 210 may restore the physiological signal measured by the electrode 243 according to the compensation value and the interfered signal in response to the compensation value matching the interfered signal in the compensation table. Specifically, the processor 210 may detect the frequency of the interfered signal, and choose a compensation value matching the frequency of the interfered signal from the compensation table to compensate the interfered signal and thereby generate the physiological signal. For example, if the interfered signal includes a signal corresponding to the frequency f1 and a signal corresponding to the frequency f2, the processor 210 may compensate the signal corresponding to the frequency f1 in the interfered signal according to the compensation value W(f1) corresponding to the frequency f1, thereby restoring the signal corresponding to the frequency f1 in the physiological signal. The processor 210 may also compensate the signal corresponding to the frequency f2 in the interfered signal according to the compensation value W(f2) corresponding to the frequency f2, thereby restoring the signal corresponding to the frequency f2 in the physiological signal.

In an embodiment, the processor 210 may start receiving the interfered signal to measure the physiological signal after receiving a complete compensation table (or finishing updating the compensation table). Referring to FIG. 3A, the processor 210 may transmit an active signal corresponding to the frequency f1 to the signal line 231 via the transmitter 221 in the time period T1, so as to obtain the compensation value corresponding to the frequency f1. Similarly, the processor 210 may transmit an active signal corresponding to the frequency f2 to the signal line 231 via the transmitter 221 in the time period T2, so as to obtain the compensation value corresponding to the frequency f2. After obtaining the compensation value corresponding to the frequency f1 and the compensation value corresponding to the frequency f2, the processor 210 may receive the interfered signal via the receiver 223 in the time period T3. The interfered signal corresponds to the physiological signal measured by the electrode 243, and the time period T3 may be after the time period T1 and the time period T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 3B, the processor 210 may transmit an active signal corresponding to the frequency range F1 to the signal line 231 via the transmitter 221 in the time period T1, so as to obtain the compensation value corresponding to the frequency range F1. Similarly, the processor 210 may transmit an active signal corresponding to the frequency range F2 to the signal line 231 via the transmitter 221 in the time period T2, so as to obtain the compensation value corresponding to the frequency range F2. After obtaining the compensation value corresponding to the frequency range F1 and the compensation value corresponding to the frequency range F2, the processor 210 may receive the interfered signal via the receiver 223 in the time period T3. The interfered signal corresponds to the physiological signal measured by the electrode 243, and the time period T3 may be after the time period T1 and the time period T2. In an embodiment, the frequency range F1 or the frequency range F2 may be a frequency range between 5 Hz to 1500 kHz, and the frequency range F1 and the frequency range F2 may be different from each other.

In an embodiment, the processor 210 may receive the interfered signal to measure the physiological signal while receiving (or updating) the compensation values of the compensation table. Referring to FIG. 4A, the processor 210 may transmit an active signal corresponding to the frequency f1 to the signal line 231 via the transmitter 221 in the time period T1, so as to obtain the compensation value corresponding to the frequency f1. Similarly, the processor 210 may transmit an active signal corresponding to the frequency f2 to the signal line 231 via the transmitter 221 in the time period T2, so as to obtain the compensation value corresponding to the frequency f2. The processor 210 may receive the interfered signal via the receiver 223 in the time period T3. The interfered signal corresponds to the physiological signal measured by the electrode 243, and the time period T3 may be between the time period T1 and the time period T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 4B, the processor 210 may transmit an active signal corresponding to the frequency range F1 to the signal line 231 via the transmitter 221 in the time period T1, so as to obtain the compensation value corresponding to the frequency range F1. Similarly, the processor 210 may transmit an active signal corresponding to the frequency range F2 to the signal line 231 via the transmitter 221 in the time period T2, so as to obtain the compensation value corresponding to the frequency range F2. The processor 210 may receive the interfered signal via the receiver 223 in the time period T3. The interfered signal corresponds to the physiological signal measured by the electrode 243, and the time period T3 may be between the time period T1 and the time period T2. In an embodiment, the frequency range F1 or the frequency range F2 may be a frequency range between 5 Hz to 1500 kHz, and the frequency range F1 and the frequency range F2 may be different from each other.

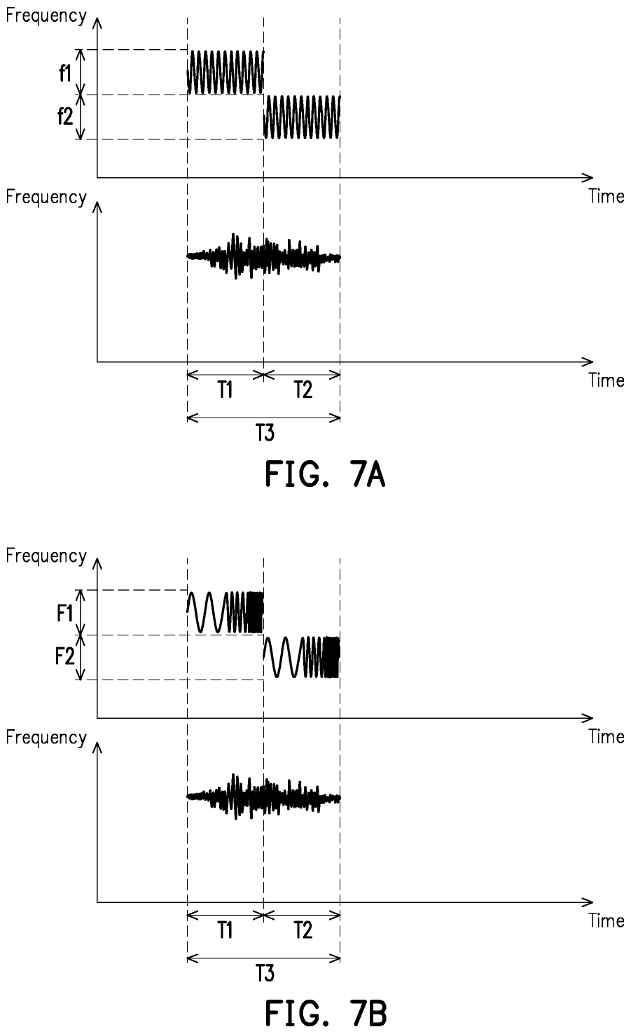

In an embodiment, the processor 210 may receive the interfered signal to measure the physiological signal while simultaneously receiving (or updating) the compensation table. Therefore, the processor 210 may measure the physiological signal when the generation or updating of the compensation table has not been finished, thereby reducing the waiting time required in measuring the physiological signal. If a compensation value corresponding to a specific frequency has not been updated, the processor 210 may restore the physiological signal according to an existing compensation value corresponding to the specific frequency. FIGS. 7A and 7B are schematic diagrams illustrating obtaining a compensation value while simultaneously measuring a physiological signal according to an embodiment of the disclosure. Referring to FIG. 7A, the processor 210 may transmit an active signal corresponding to the frequency f1 to the signal line 231 via the transmitter 221 in the time period T1, so as to obtain the compensation value corresponding to the frequency f1. Similarly, the processor 210 may transmit an active signal corresponding to the frequency f2 to the signal line 231 via the transmitter 221 in the time period T2, so as to obtain the compensation value corresponding to the frequency f2. The processor 210 may receive the interfered signal via the receiver 223 in the time period T3. The interfered signal corresponds to the physiological signal measured by the electrode 243, and the time period T3 may be entirely or partially overlapped with the time period T1 or T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 7B, the processor 210 may transmit an active signal corresponding to the frequency range F1 to the signal line 231 via the transmitter 221 in the time period T1, so as to obtain the compensation value corresponding to the frequency range F1. Similarly, the processor 210 may transmit an active signal corresponding to the frequency range F2 to the signal line 231 via the transmitter 221 in the time period T2, so as to obtain the compensation value corresponding to the frequency range F2. The processor 210 may receive the interfered signal via the receiver 223 in the time period T3. The interfered signal corresponds to the physiological signal measured by the electrode 243, and the time period T3 may be entirely or partially overlapped with the time period T1 or T2. In an embodiment, the frequency range F1 or the frequency range F2 may be a frequency range between 5 Hz to 1500 kHz, and the frequency range F1 and the frequency range F2 may be different from each other.

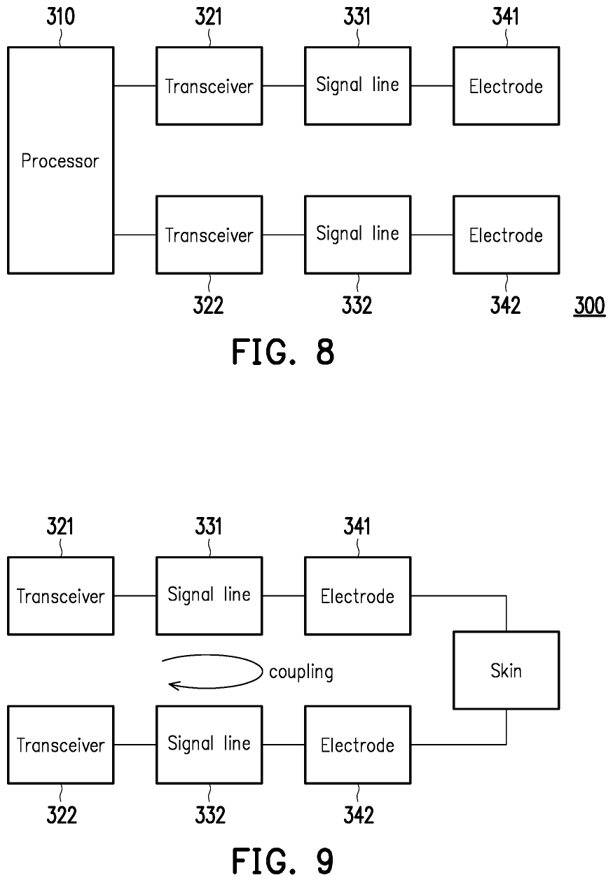

FIG. 8 is a schematic diagram illustrating an electronic device 300 for signal interference compensation according to an embodiment of the disclosure. The electronic device 300 may include a processor 310, a transceiver 321, a transceiver 322, a signal line 331, a signal line 332, an electrode 341, and an electrode 342.

The processor 310 is, for example, a central processing unit (CPU), or other programmable general-purpose/specific purpose micro control units (MCUs), microprocessors, digital signal processors (DSPs), programmable controllers, application specific integrated circuits (ASICs), graphics processing units (GPUs), image signal processors (ISPs), image processing units (IPUs), arithmetic logic units (ALUs), complex programmable logic devices (CPLDs), field programmable gate arrays (FPGAs), other similar components, or a combination thereof. The processor 310 may be electrically connected to the transceiver 321 and the transceiver 322. The processor 310 may include a storage medium. The storage medium may be, for example, any type of fixed or removable random access memories (RAMs), read-only memories (ROMs), flash memories, hard disk drives (HDDs), solid state drives (SSDs), other similar components, or a combination thereof.

The transceiver 321 may be electrically connected to the signal line 331, and may be configured to transmit a signal to the signal line 331 or receive a signal from the signal line 331. The transceiver 321 may be further capable of, for example, low noise amplification, impedance matching, frequency mixing, up or down frequency conversion, filtering, amplification, and similar operations.

The transceiver 322 may be electrically connected to the signal line 332, and may be configured to transmit a signal to the signal line 332 or receive a signal from the signal line 332. The transceiver 322 may be further capable of, for example, low noise amplification, impedance matching, frequency mixing, up or down frequency conversion, filtering, amplification, and similar operations.

The electrode 341 may be electrically connected with the signal line 331. When contacting the user's skin, the electrode 341 may measure a physiological signal from the skin. The physiological signal is, for example, an EMG signal.

The electrode 342 may be electrically connected with the signal line 332. When contacting the user's skin, the electrode 342 may measure a physiological signal from the skin.

FIG. 9 is a schematic diagram illustrating obtaining a compensation value and restoring the physiological signal by the electronic device 300 according to an embodiment of the disclosure. The signal line 331 may be wound together with the signal line 332, and may be coupled with the signal line 331. The processor 310 may transmit an active signal at a specific frequency to the signal line 331 via the transceiver 321. The signal line 332 wound with the signal line 331 may generate a coupling signal corresponding to the active signal. The processor 310 may receive the coupling signal via the transceiver 322, and calculate a compensation value according to the coupling signal.

The processor 310 may obtain a plurality of compensation values in correspondence with different frequencies or different frequency ranges, so as to generate a compensation table according to the compensation values. Specifically, the active signal may include the first signal corresponding to the first frequency (or the first frequency range) and the second signal corresponding to the second frequency (or the second frequency range). In an embodiment, the processor 310 may transmit the first signal to the signal line 331 via the transceiver 321 in the first time period. The signal line 332 may generate the first coupling signal in correspondence with the first signal. The processor 310 may receive the first coupling signal via the transceiver 322, and calculate the first compensation value according to the first signal and the first coupling signal. In addition, the first compensation value corresponds to the first frequency. Besides, the processor 310 may transmit the second signal to the signal line 331 via the transceiver 321 in the second time period different from the first time period. The signal line 332 may generate the second coupling signal in correspondence with the second signal. The processor 310 may receive the second coupling signal via the transceiver 322, and calculate the second compensation value according to the second signal and the second coupling signal. The second compensation value corresponds to the second frequency different from the first frequency. The processor 310 may obtain N compensation values respectively corresponding to different frequencies (or frequency ranges), and generate the compensation table as shown in Table 1 according to the N compensation values. If the storage medium of the processor 310 already stores an existing compensation table, the processor 310 may update the existing compensation table according to the N compensation values.

After obtaining the compensation table, the processor 310 may use the compensation table to restore the measured physiological signal. Specifically, the electrode 342 may contact the user's skin to measure the physiological signal. The physiological signal may be transmitted to the transceiver 322 via the signal line 332. However, under the influence of crosstalk, the physiological signal may be turned into an interfered signal when passing through the signal line 332.

After the processor 310 receives the interfered signal via the transceiver 322, the processor 310 may restore the physiological signal measured by the electrode 342 according to the compensation value and the interfered signal in response to the compensation value matching the interfered signal in the compensation table. Specifically, the processor 310 may detect the frequency of the interfered signal, and choose a compensation value matching the frequency of the interfered signal from the compensation table to compensate the interfered signal and thereby generate the physiological signal. For example, if the interfered signal includes a signal corresponding to the frequency f1 and a signal corresponding to the frequency f2, the processor 310 may compensate the signal corresponding to the frequency f1 in the interfered signal according to the compensation value W(f1) corresponding to the frequency f1, thereby restoring the signal corresponding to the frequency f1 in the physiological signal. The processor 310 may also compensate the signal corresponding to the frequency f2 in the interfered signal according to the compensation value W(f2) corresponding to the frequency f2, thereby restoring the signal corresponding to the frequency f2 in the physiological signal.

In an embodiment, the processor 310 may transmit the first signal to the signal line 332 via the transceiver 322 in the first time period. The signal line 331 may generate the first coupling signal in correspondence with the first signal. The processor 310 may receive the first coupling signal via the transceiver 321, and calculate the first compensation value according to the first signal and the first coupling signal. In addition, the first compensation value corresponds to the first frequency. Besides, the processor 310 may transmit the second signal to the signal line 332 via the transceiver 322 in the second time period different from the first time period. The signal line 331 may generate the second coupling signal in correspondence with the second signal. The processor 310 may receive the second coupling signal via the transceiver 321, and calculate the second compensation value according to the second signal and the second coupling signal. The second compensation value corresponds to the second frequency different from the first frequency. The processor 310 may obtain N compensation values respectively corresponding to different frequencies (or frequency ranges), and generate the compensation table as shown in Table 1 according to the N compensation values. If the storage medium of the processor 310 already stores an existing compensation table, the processor 310 may update the existing compensation table according to the N compensation values.

After obtaining the compensation table, the processor 310 may use the compensation table to restore the measured physiological signal. Specifically, the electrode 341 may contact the user's skin to measure the physiological signal. The physiological signal may be transmitted to the transceiver 321 via the signal line 331. However, under the influence of crosstalk, the physiological signal may be turned into an interfered signal when passing through the signal line 331.

After the processor 310 receives the interfered signal via the transceiver 321, the processor 310 may restore the physiological signal measured by the electrode 341 according to the compensation value and the interfered signal in response to the compensation value matching the interfered signal in the compensation table. Specifically, the processor 310 may detect the frequency of the interfered signal, and choose a compensation value matching the frequency of the interfered signal from the compensation table to compensate the interfered signal and thereby generate the physiological signal. For example, if the interfered signal includes a signal corresponding to the frequency f1 and a signal corresponding to the frequency f2, the processor 310 may compensate the signal corresponding to the frequency f1 in the interfered signal according to the compensation value W(f1) corresponding to the frequency f1, thereby restoring the signal corresponding to the frequency f1 in the physiological signal. The processor 310 may also compensate the signal corresponding to the frequency f2 in the interfered signal according to the compensation value W(f2) corresponding to the frequency f2, thereby restoring the signal corresponding to the frequency f2 in the physiological signal.

In an embodiment, the processor 310 may start receiving the interfered signal to measure the physiological signal after receiving a complete compensation table (or finishing updating the compensation table). Referring to FIG. 3A, the processor 310 may transmit an active signal corresponding to the frequency f1 to the signal line 331 via the transceiver 321 in the time period T1, so as to obtain the compensation value corresponding to the frequency f1. Similarly, the processor 310 may transmit an active signal corresponding to the frequency f2 to the signal line 331 via the transceiver 321 in the time period T2, so as to obtain the compensation value corresponding to the frequency f2. After obtaining the compensation value corresponding to the frequency f1 and the compensation value corresponding to the frequency f2, the processor 310 may receive the interfered signal via the transceiver 322 in the time period T3. The interfered signal corresponds to the physiological signal measured by the electrode 342, and the time period T3 may be after the time period T1 and the time period T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 3B, the processor 310 may transmit an active signal corresponding to the frequency range F1 to the signal line 331 via the transceiver 321 in the time period T1, so as to obtain the compensation value corresponding to the frequency range F1. Similarly, the processor 310 may transmit an active signal corresponding to the frequency range F2 to the signal line 331 via the transceiver 321 in the time period T2, so as to obtain the compensation value corresponding to the frequency range F2. After obtaining the compensation value corresponding to the frequency range F1 and the compensation value corresponding to the frequency range F2, the processor 310 may receive the interfered signal via the transceiver 322 in the time period T3. The interfered signal corresponds to the physiological signal measured by the electrode 342, and the time period T3 may be after the time period T1 and the time period T2. In an embodiment, the frequency range F1 or the frequency range F2 may be a frequency range between 5 Hz to 1500 kHz, and the frequency range F1 and the frequency range F2 may be different from each other.

In an embodiment, the processor 310 may receive the interfered signal to measure the physiological signal while receiving (or updating) the compensation values of the compensation table. Referring to FIG. 4A, the processor 310 may transmit an active signal corresponding to the frequency f1 to the signal line 331 via the transceiver 321 in the time period T1, so as to obtain the compensation value corresponding to the frequency f1. Similarly, the processor 310 may transmit an active signal corresponding to the frequency f2 to the signal line 331 via the transceiver 321 in the time period T2, so as to obtain the compensation value corresponding to the frequency f2. The processor 310 may receive the interfered signal via the transceiver 322 in the time period T3. The interfered signal corresponds to the physiological signal measured by the electrode 342, and the time period T3 may be between the time period T1 and the time period T2. In an embodiment, the frequency f1 or the frequency f2 may be 5 Hz, 6 Hz, 7 Hz, . . . , or 1500 kHz, etc., and the frequency f1 and the frequency f2 may be different from each other.

Referring to FIG. 4B, the processor 310 may transmit an active signal corresponding to the frequency range F1 to the signal line 331 via the transceiver 321 in the time period T1, so as to obtain the compensation value corresponding to the frequency range F1. Similarly, the processor 310 may transmit an active signal corresponding to the frequency range F2 to the signal line 331 via the transceiver 321 in the time period T2, so as to obtain the compensation value corresponding to the frequency range F2. The processor 310 may receive the interfered signal via the transceiver 322 in the time period T3. The interfered signal corresponds to the physiological signal measured by the electrode 342, and the time period T3 may be between the time period T1 and the time period T2. In an embodiment, the frequency range F1 or the frequency range F2 may be a frequency range between 5 Hz to 1500 kHz, and the frequency range F1 and the frequency range F2 may be different from each other.

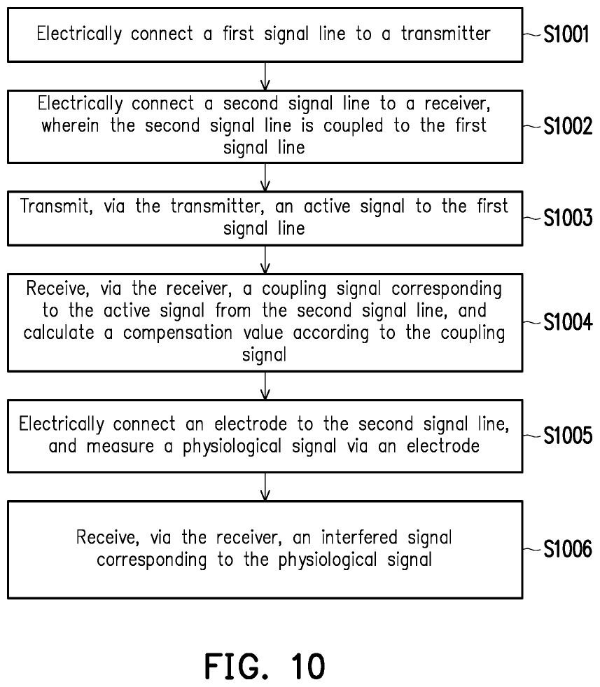

FIG. 10 is a flowchart illustrating the first method for signal interference compensation according to an embodiment of the disclosure. The first method may be carried out by the electronic device 100 shown in FIG. 1. In Step S1001, the first signal line is electrically connected to the transmitter. In Step S1002, the second signal line is electrically connected to the receiver, and the second signal line is coupled to the first signal line. In Step S1003, the active signal is transmitted to the first signal line via the transmitter. In Step S1004, the coupling signal corresponding to the active signal is received from the second signal line via the receiver, and the compensation value is calculated according to the coupling signal. In Step S1005, the electrode is electrically connected to the second signal line, and the physiological signal is measured via the electrode. In Step S1006, the interfered signal corresponding to the physiological signal is received via the receiver, and the physiological signal is restored according to the compensation value and the interfered signal in response to the compensation value matching the interfered signal.

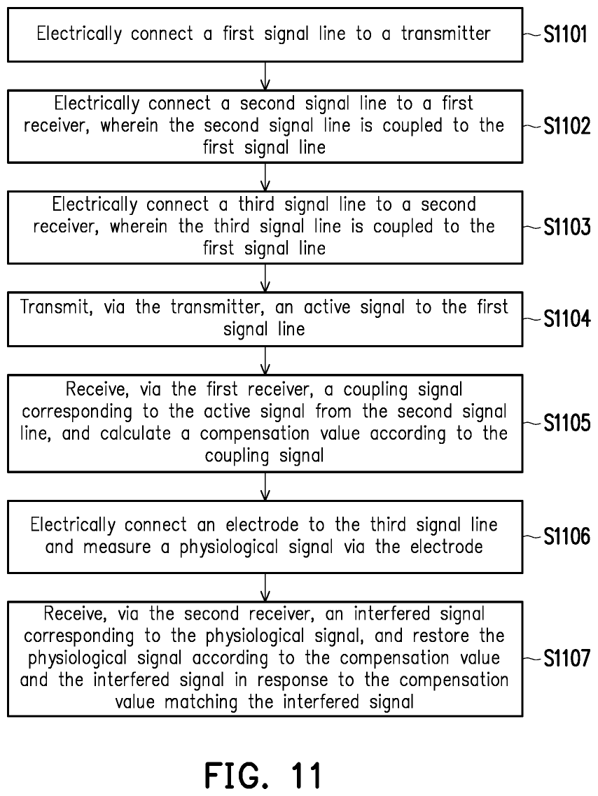

FIG. 11 is a flowchart illustrating the second method for signal interference compensation according to an embodiment of the disclosure. The second method may be carried out by the electronic device 200 shown in FIG. 5. In Step S1101, the first signal line is electrically connected to the transmitter. In Step S1102, the second signal line is electrically connected to the first receiver, and the second signal line is coupled to the first signal line. In Step S1103, the third signal line is electrically connected to the second receiver, and the third signal line is coupled to the first signal line. In Step S1104, the active signal is transmitted to the first signal line via the transmitter. In Step S1105, the coupling signal corresponding to the active signal is received from the second signal line via the first receiver, and the compensation value is calculated according to the coupling signal. In Step S1106, the electrode is electrically connected to the third signal line, and the physiological signal is measured via the electrode. In Step S1107, the interfered signal corresponding to the physiological signal is received via the second receiver, and the physiological signal is restored according to the compensation value and the interfered signal in response to the compensation value matching the interfered signal.

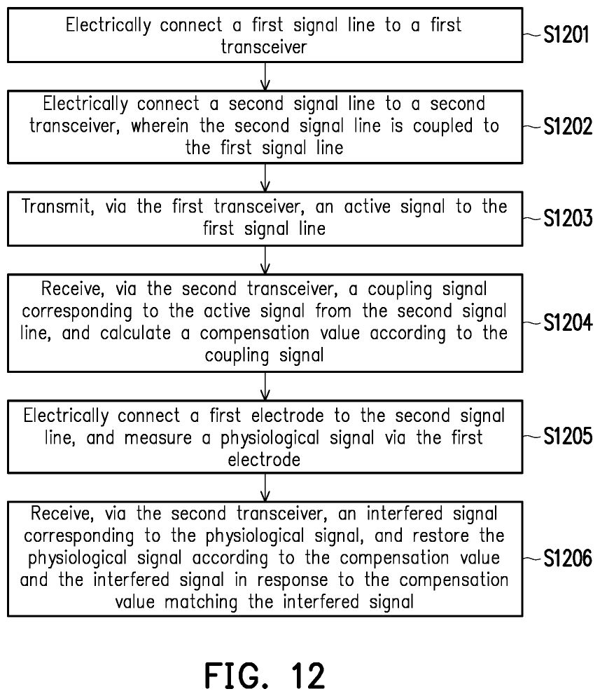

FIG. 12 is a flowchart illustrating the third method for signal interference compensation according to an embodiment of the disclosure. The third method may be carried out by the electronic device 300 shown in FIG. 8. In Step S1201, the first signal line is electrically connected to the first transceiver. In Step S1202, the second signal line is electrically connected to the second transceiver, and the second signal line is coupled to the first signal line. In Step S1203, the active signal is transmitted to the first signal line via the first transceiver. In Step S1204, the coupling signal corresponding to the active signal is received from the second signal line via the second transceiver, and the compensation value is calculated according to the coupling signal. In Step S1205, the first electrode is electrically connected to the second signal line, and the physiological signal is measured via the first electrode. In Step S1206, the interfered signal corresponding to the physiological signal is received via the second transceiver, and the physiological signal is restored according to the compensation value and the interfered signal in response to the compensation value matching the interfered signal.

In view of the foregoing, the electronic device according to the embodiments of the disclosure is capable of transmitting an active signal to obtain the compensation values corresponding to different frequencies, and each of the compensation values serves to compensate for the crosstalk influence on the signal line. When the measured physiological signal is turned into the interfered signal due to crosstalk, the electronic device is capable of restoring the interfered signal according to the compensation value, so as to calculate the physiological signal without distortion. Therefore, even if multiple signal lines are wound together, the electronic device is still capable of eliminating the distortion of the physiological signal measured by each signal line. The physiological signal may be measured after all the compensation values are obtained or between different time periods for obtaining different compensation values. Furthermore, the measurement of the physiological signal may also be carried out simultaneously with the obtaining of the compensation values. Accordingly, even if the electronic device needs to carry out the step of obtaining compensation values for multiple times, the measurement of the physiological signal is not delayed or interrupted.

It will be apparent to those skilled in the art that various modifications and variations can be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. An electronic device for signal interference compensation, the electronic device comprising:
a transmitter;
a first signal line, electrically connected to the transmitter;
a receiver;
a second signal line, electrically connected to the receiver and coupled to the first signal line;
an electrode, electrically connected to the second signal line and measuring a physiological signal; and
a processor, electrically connected to the transmitter and the receiver, and is configured to:
transmit, via the transmitter, an active signal to the first signal line;
receive, via the receiver, a coupling signal corresponding to the active signal from the second signal line, and calculate a compensation value according to the coupling signal; and
receive, via the receiver, an interfered signal corresponding to the physiological signal, and restore the physiological signal according to the compensation value and the interfered signal in response to the compensation value matching the interfered signal.

2. The electronic device as claimed in claim 1, wherein the active signal comprises a first signal corresponding to a first frequency and a second signal corresponding to a second frequency, wherein the compensation value comprises a first compensation value corresponding to the first frequency and a second compensation value corresponding to the second frequency, the processor transmits the first signal in a first time period to obtain the first compensation value and transmits the second signal in a second time period to obtain the second compensation value, and the first time period is different from the second time period.

3. The electronic device as claimed in claim 2, wherein the processor detects a frequency of the interfered signal and restores the physiological signal according to the first compensation value in response to the frequency matching the first compensation value.

4. The electronic device as claimed in claim 2, wherein the processor receives the interfered signal in a third time period, and the third time period is after the first time period and the second time period.

5. The electronic device as claimed in claim 2, wherein the processor receives the interfered signal in a third time period, and the third time period is between the first time period and the second time period.

6. An electronic device for signal interference compensation, the electronic device comprising:
a transmitter;
a first signal line, electrically connected to the transmitter;
a first receiver;
a second signal line, electrically connected to the first receiver and coupled to the first signal line;
a second receiver;
a third signal line, electrically connected to the second receiver and coupled to the first signal line;
an electrode, electrically connected to the third signal line and measuring a physiological signal; and
a processor, electrically connected to the transmitter, the first receiver, and the second receiver, and is configured to:
transmit, via the transmitter, an active signal to the first signal line;
receive, via the first receiver, a coupling signal corresponding to the active signal from the second signal line, and calculate a compensation value according to the coupling signal; and
receive, via the second receiver, an interfered signal corresponding to the physiological signal, and restore the physiological signal according to the compensation value and the interfered signal in response to the compensation value matching the interfered signal.

7. The electronic device as claimed in claim 6, wherein the active signal comprises a first signal corresponding to a first frequency and a second signal corresponding to a second frequency, wherein the compensation value comprises a first compensation value corresponding to the first frequency and a second compensation value corresponding to the second frequency, the processor transmits the first signal in a first time period to obtain the first compensation value and transmits the second signal in a second time period to obtain the second compensation value, and the first time period is different from the second time period.

8. The electronic device as claimed in claim 7, wherein the processor detects a frequency of the interfered signal and restores the physiological signal according to the first compensation value in response to the frequency matching the first compensation value.

9. The electronic device as claimed in claim 7, wherein the processor receives the interfered signal in a third time period, and the third time period is after the first time period and the second time period.

10. The electronic device as claimed in claim 7, wherein the processor receives the interfered signal in a third time period, and the third time period is between the first time period and the second time period.

11. The electronic device as claimed in claim 7, wherein the processor receives the interfered signal in a third time period, and the third time period is overlapped with at least one of the first time period and the second time period.

12. An electronic device for signal interference compensation, the electronic device comprising:
a first transceiver;
a first signal line, electrically connected to the first transceiver;
a second transceiver;
a second signal line, electrically connected to the second transceiver and coupled to the first signal line;
a first electrode, electrically connected to the second signal line and measuring a physiological signal; and
a processor, electrically connected to the first transceiver and the second transceiver, and is configured to:
transmit, via the first transceiver, an active signal to the first signal line;
receive, via the second transceiver, a coupling signal corresponding to the active signal from the second signal line, and calculate a compensation value according to the coupling signal; and
receive, via the second transceiver, an interfered signal corresponding to the physiological signal, and restore the physiological signal according to the compensation value and the interfered signal in response to the compensation value matching the interfered signal.

13. The electronic device as claimed in claim 12, wherein the active signal comprises a first signal corresponding to a first frequency and a second signal corresponding to a second frequency, wherein the compensation value comprises a first compensation value corresponding to the first frequency and a second compensation value corresponding to the second frequency, the processor transmits the first signal in a first time period to obtain the first compensation value and transmits the second signal in a second time period to obtain the second compensation value, and the first time period is different from the second time period.

14. The electronic device as claimed in claim 13, wherein the processor detects a frequency of the interfered signal and restores the physiological signal according to the first compensation value in response to the frequency matching the first compensation value.

15. The electronic device as claimed in claim 13, wherein the processor receives the interfered signal in a third time period, and the third time period is after the first time period and the second time period.

16. The electronic device as claimed in claim 13, wherein the processor receives the interfered signal in a third time period, and the third time period is between the first time period and the second time period.

17. The electronic device as claimed in claim 12, further comprising:
a second electrode, electrically connected to the first signal line and measuring a second physiological signal, wherein the processor is further configured to:
transmit, via the second transceiver, a second active signal to the second signal line;
receive, via the first transceiver, a second coupling signal corresponding to the second active signal from the first signal line, and calculate a second compensation value according to the second coupling signal; and
receive, via the first transceiver, a second interfered signal corresponding to the second physiological signal, and restore the second physiological signal according to the second compensation value and the second interfered signal in response to the second compensation value matching the second interfered signal.
